(19) United States
(12) Patent Application Publication
(10) Pub. No.: US 2006/0193804 A1
(43) Pub. Date: Aug. 31, 2006

(54) Haircare use of cyclic amine derivatives

(75) Inventor: Alexandre Cavezza, Les Pavillons Sous Bois (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/360,518

(22) Filed: Feb. 24, 2006

Related U.S. Application Data
(60) Provisional application No. 60/685,878, filed on Jun. 1, 2005.

(30) Foreign Application Priority Data
Feb. 24, 2005 (FR) 05 50512

(51) Int. Cl. A61K 8/00

(52) U.S. Cl. 424/70.1

(57) ABSTRACT

The present invention relates to a process for treating human keratin fibers and/or the skin from which the said fibers emerge, in order to induce and/or stimulate the growth of the human keratin fibers and/or to stop their loss and/or to increase their density, comprising at least the application, to the human keratin fibers and/or the skin, of a cosmetic composition comprising an effective amount of at least one piperidine derivative of general formula (I), 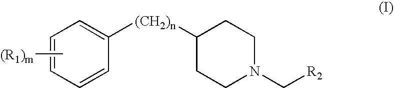 in which:
R₁ denotes a halogen or a radical chosen from a linear or branched C₁-C₆ alkyl radical, a group OR, a group NRR′, a CF₃ group, a group NHCOR or a group CONRR′;
R₂ denotes a linear or branched C₁-C₂₀ alkyl or alkenyl radical, the hydrocarbon-based chain of which may be optionally interrupted with a —CO— function optionally substituted with at least one group OR, COOR, NRR′, NHCOR or CONRR′ and/or with a phenyl group optionally substituted with one or more radicals R₁;
   with R and R′ denoting, independently of each other, a hydrogen atom or a linear or branched C₁-C₆ alkyl radical, m represents an integer ranging from 0 to 5; and n represents an integer ranging from 0 to 5. or a salt or isomer thereof, leaving the said composition in contact with the keratin fibers and/or the skin from which the fibers emerge, and optionally rinsing the keratin fibers and/or the skin.

### CROSS-REFERENCE TO RELATED APPLICATIONS

This non provisional application claims the benefit of French Application No. 05 50512 filed on Feb. 24, 2005 and U.S. Provisional Application No. 60/685,878 filed on Jun. 1^{st}, 2005.

### BACKGROUND

The present invention relates to the use of a family of cyclic amine derivatives for example as active agents for counteracting hair loss and/or for treating alopecia.

Hair growth and hair renewal are mainly determined by the activity of the hair follicles and of their surrounding matrix. This activity is cyclical and comprises essentially three phases, namely the anagenic phase, the catagenic phase and the telogenic phase.

The anagenic phase (active phase or growth phase), which lasts several years, corresponds to the active phase or growth phase of the hair.

The catagenic phase that follows the anagenic phase is, on the other hand, very short and transient, lasting a few weeks. During this phase, the hair undergoes a change, the follicle becomes atrophied and its dermal implantation appears higher and higher.

The terminal phase or telogenic phase, which lasts a few months, corresponds to a resting phase of the follicle and the hair ends up by falling out. At the end of this resting period, a new follicle is regenerated in situ and another cycle begins.

The head of hair is thus under permanent renewal, and, out of the approximately 150 000 hairs that make up a head of hair, about 10% are at rest and will be replaced within a few months. The natural loss or falling-out of the hair may be estimated, on average, as being a few hundred hairs per day for a normal physiological state. This process of permanent physical renewal undergoes a natural change during ageing; the hairs become finer and their cycles shorter.

In adulthood, the vascular system of the skin is complete and no longer changes, except for in the hair follicles, where it undergoes large changes with each hair cycle. Specifically, the hair follicles are a richly innervated and highly vascularized cutaneous structure. The phenomenon of development of capillary circulation in the hair follicles is known as angiogenesis. At the start of each anagenic phase, it is necessary to develop high activation of angiogenesis in order to redevelop the perifollicular vascular capillary network. The involution of this capillary network and the disappearance of the blood vessels of the dermal papilla go hand in hand with the change of phase and the passage into the catagenic phase. At this stage, the blood capillaries collapse and disappear.

In parallel, in the alopecic areas, a perifollicular fibrosis becomes established, the follicles reduce in size cycle after cycle and the specific vascularization of the bulbs gradually diminishes.

The phenomenon of angiogenesis observed during the anagenic phase is dependent on many trophic factors, cytokines or other biologically active molecules provided by the blood circulation or produced locally, in particular by the fibroblasts of the dermal papilla or the keratinocytes of the hair bulb. Among these trophic factors, mention may be made of endothelial cell growth factor (also known as vascular endothelial growth factor (VEGF)). This factor is essential for angiogenesis and increases the vascular permeability. Studies have shown that the expression of this factor was increased during the anagenic phase of the hair cycle. Thus, this factor contributes towards maintaining functional capillary vascularization around the hair follicle and especially at the base of the bulb and of the dermal papilla, and also towards supplying nutrients required for good growth of the hair.

The perifollicular capillary circulation thus plays a fundamental role in the process of hair growth by supplying the factors and nutrients required for the growth of this follicle.

Consequently, any impairment in the perifollicular capillary circulation will result in a reduction in the supply of nutrients and gases (especially oxygen) required for hair growth, leading to disturbances in the growth of the hair and the gradual establishment of alopecia.

Thus, in certain dermatoses of the scalp with an inflammatory component, for instance psoriasis or seborrhoeic dermatitis, hair loss may be greatly accentuated and the follicle renewal cycles may be highly disrupted.

Moreover, pregnancy (post-partum), states of dietary denutrition or malnutrition, physiological stress or dietary imbalance, or alternatively states of asthenia or of hormonal dysfunction, as may be the case during or at the terminal stage of the menopause, may also result in substantial temporary or permanent loss and/or impairment of hair. It may also be a case of loss or impairment of the hair related to seasonal phenomena.

It may also be a matter of alopecia, which is essentially due to a disturbance in hair renewal, resulting, in a first stage, in acceleration of the frequency of the cycles to the detriment of the quality of the hair, and then of their quantity. This then results in a gradual impoverishment of the head of hair and in gradual thinning of the hair together with isolation of the bulbs due to progressive thickening of the perifollicular collagen matrix and of the outer connective sheath. Revascularization is thus made more difficult cycle after cycle. The successive growth cycles result in hairs that are finer and finer and shorter and shorter, gradually transforming into an unpigmented down. Certain areas are preferentially affected, especially the temporal or frontal lobes in men, and a diffuse alopecia of the crown of the head in women.

The term alopecia also covers a whole family of afflictions of hair follicles whose final consequence is the permanent, partial or general loss of the hair. This is more particularly termed androgenic alopecia. In a large number of cases, early loss of hair occurs in genetically predisposed individuals; this is then termed andro-chrono-genetic alopecia. This form of alopecia especially affects men.

Compositions for suppressing or reducing alopecia, and for example for inducing or stimulating hair growth or reducing hair loss have been sought for many years in the cosmetics and pharmaceuticals industries. One of the routes explored is for example that of maintenance of the vascularization around the hair follicle.

In general, any factor that results in an increase in the blood supply to the hair follicles, either by activating angiogenesis, combating its regression or acting on the capillaries to limit their constriction, will have a beneficial effect on the energy supply required for good growth of these follicles.

One method for controlling loss-preventing/regrowth-promoting activity of a compound on keratin fibers is directed, precisely, towards testing the efficacy of the said compound as regards the decontraction of muscles. Those skilled in the art know that the effects following this decontraction, i.e. a reduction in the thickness of the collagen matrix around the hair bulbs and/or an increase in vascularization around the hair bulbs, are predictive of a beneficial effect of the test compound in terms of loss-preventing and/or regrowth-promoting action on keratin fibers.

Thus, one of the compounds known to maintain perifollicular vascularization is verapamil, which is a powerful type L calcium-channel antagonist. Verapamil and other calcium-channel antagonists such as diltiazem and nifedipine are described as being active in the treatment of hair loss, in particular as a result of their effects on capillary circulation (cf. the documents by Shiseido JP 88/062680 and Coppe J. BE/89/000305).

In addition, documents exist describing the use of NO (nitrogen monoxide) donors for application to the scalp, to stimulate hair growth by acting on the capillary circulation of the scalp. Thus, the patent by Proctor (EP 0 327 263) describes the use of compounds producing the NO radical, in combination with reducing agents, antioxidants and hydroxyl-radical scavengers. Another patent by E. Fossel (WO 99/13717) describes the use of arginine and derivatives thereof as an NO-synthase substrate for the in vivo formation of NO and their use (inter alia) in the treatment of alopecia. Another patent by Shiseido (JP-A-07 316 023) also describes the use of arginine and its derivatives in the treatment of alopecia.

However, these known substances have adverse effects. For example, they have multiple activities, which may disrupt the ionic and physiological equilibrium of the skin cells. In other words, their multiple activity makes it difficult to control their action on cells.

More recently, patent application EP 1 506 767 has described a particular family of carbonyl amines that have a beneficial effect in terms of improving the vascularization of hair follicles, thus promoting hair growth.

The present invention results more particularly from the observation by the inventors that a particular family of piperidine derivatives also has beneficial activity with regard to the hair follicles, their peripheral region and/or the hair.

Among the compounds used according to the invention, some have already been described as calcium-channel inhibitors in patent applications EP-0 542 846 and JP-61 027 963 and in patent U.S. Pat. No. 4,952,560.

Other piperidine derivatives have been described as antibacterial agents (EP-0 308 328).

Patent application JP 62 270 514 has, in turn, proposed using a specific piperidine derivative, Ifenprodil, in haircare compositions to improve the blood circulation of the scalp.

### SUMMARY

Consequently, according to one exemplary embodiment, the present invention relates to a process for treating human keratin fibers and/or the skin from which the said fibers emerge, including the scalp and the eyelids, in order to induce and/or to stimulate the growth of human keratin fibers, such as human head hair and the eyelashes and/or to reduce their loss and/or to increase their density, comprising at least the application, to the human keratin fibers and/or the skin, of a cosmetic composition comprising an effective amount of at least one piperidine derivative of formula (I): 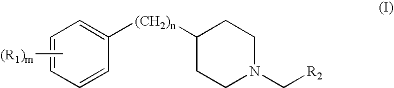
in which:

R₁ denotes a halogen or a radical chosen from a linear or branched C₁-C₆ alkyl radical, a group OR, a group NRR′, a CF₃ group, a group NHCOR or a group CONRR′;

R₂ denotes a linear or branched C₁-C₁₉ alkyl or alkenyl radical, the hydrocarbon-based chain of which may be optionally interrupted with a —CO— function and optionally substituted with at least one group OR, COOR, NRR′, NHCOR or CONRR′ and/or with a phenyl group optionally substituted with one or more radicals R₁;

with R and R′ denoting, independently of each other, a hydrogen atom or a linear or branched C₁-C₆ alkyl radical,

m represents an integer ranging from 0 to 5; and

n represents an integer ranging from 0 to 5;

or a salt and isomer thereof, leaving the said composition in contact with the keratin fibers and/or the skin from which the fibers emerge, and optionally rinsing the keratin fibers and/or the skin.

According to another exemplary embodiment, the invention relates to a process for treating disorders associated with a reduction in cutaneous capillary circulation or vascularization, for example of human hair follicles, comprising at least the application, to the human keratin fibers and/or the skin, of a cosmetic composition comprising an effective amount of at least one piperidine derivative of general formula (I) or a salt or isomer thereof, leaving the said composition in contact with the keratin fibers and/or the skin, and optionally rinsing the keratin fibers and/or the skin.

In one exemplary embodiment, the present invention relates to a care process for human hair and/or the scalp, to improve their condition and/or appearance, comprising at least the application to the hair and/or the scalp of a cosmetic composition comprising an effective amount of at least one piperidine derivative of general formula (I) or a salt or isomer thereof, leaving the said composition in contact with the hair and/or the scalp, and optionally rinsing the hair and/or the scalp.

According to one exemplary embodiment, the invention relates to a process for treating andro-chrono-genetic alopecia, comprising at least the application to the human hair and/or the scalp, of a cosmetic composition comprising an effective amount of at least one piperidine derivative of general formula (I) or a salt or isomer thereof, leaving the said composition in contact with the human hair and/or the scalp, and optionally rinsing the human hair and/or the scalp.

In another exemplary embodiment, the invention relates to a care and/or makeup process for human eyelashes, for example to improve their condition and/or their appearance, comprising at least the application to the eyelashes and/or the upper eyelids of a mascara composition comprising at least one piperadine derivative of general formula (I) or a salt or isomer thereof. This mascara composition may be applied alone or as a basecoat for a standard pigmented mascara, and may be removed like a standard pigmented mascara.

A process according to the invention may be for example a cosmetic process.

According to another exemplary embodiment, the invention relates to the use of at least one piperidine derivative of general formula (I) or a salt or isomer thereof, for the preparation of a cosmetic composition for caring for and/or treating human keratin fibers, which is intended to be used for reducing the loss of the keratin fibers and/or for increasing their density.

According to another exemplary embodiment, the invention relates to the use of at least one piperidine derivative of general formula (I) or a salt or isomer thereof, for the preparation of a cosmetic composition for caring for and/or treating human keratin fibers, which is intended to be used for inducing and/or stimulating the growth of the keratin fibers.

The human keratin fibers to which the invention applies are for example head hair, the nails, the eyebrows, the eyelashes, beard hair, moustache hair and pubic hair. According to one exemplary embodiment, the invention applies to human head hair and/or eyelashes.

According to another exemplary embodiment, the invention relates to the use of at least one piperidine derivative of general formula (I) or a salt or isomer thereof, for the preparation of a human haircare composition for inducing and/or stimulating growth of the hairFor example, this composition makes it possible to keep the head of hair in good condition and/or to combat natural hair loss, for example in men.

According to another exemplary embodiment, the invention relates to the use of at least one piperidine derivative of general formula (I) or a salt or isomer thereof, for the preparation of a cosmetic composition for human hair care intended to be used for treating andro-chrono-genetic alopecia.

According to another exemplary embodiment, the invention relates to the use of at least one piperidine derivative of general formula (I) or a salt or isomer thereof for the preparation of a care and/or makeup composition for human eyelashes, which is intended for inducing and/or stimulating the growth of the eyelashes and/or for increasing their density. This composition thus makes it possible to keep the eyelashes in good condition and/or to improve their condition and/or their appearance.

### DETAILED DESCRIPTION OF EMBODIMENTS

In formula (I), the alkyl groups may be chosen for example, depending on the case, from methyl, ethyl, n-propyl, isopropyl, n-butyl, isobutyl, tert-butyl, pentyl, hexyl, heptyl, octyl, nonyl, decyl, undecyl, dodecyl, myristyl, palmityl, stearyl and arachidyl groups.

In addition, in the context of the present invention, the term “alkenyl” means radicals that may comprise one or more conjugated or non-conjugated double bonds. They may be chosen especially, depending on the case, from vinyl, allyl, butenyl and pentenyl groups.

Certain compounds of general formula (I) may exist in different isomeric forms such as enantiomers, diastereoisomers, geometrical isomers or tautomers. The present invention covers all these isomers and their mixture in all proportions.

Salts of the compound of formula (I) that may be mentioned include the salts obtained by addition of the compound of formula (I) with a mineral acid chosen for example from hydrochloric acid, sulfuric acid and phosphoric acid, or with an organic acid chosen for example from acetic acid, propionic acid, succinic acid, fumaric acid, maleic acid, lactic acid, glycolic acid, citric acid and tartaric acid. The salts include metal salts such as alkali metal or alkaline-earth metal salts.

The compounds according to the invention may also exist in the form of solvates and for example hydrates, which also form part of the field of the invention.

According to one exemplary embodiment, the piperidine derivative according to the invention is such that at least one and for example all of the following conditions are satisfied:

m ranges from 0 to 3 and is for example equal to 0,

n is equal to 0, 1, 2 or 3, and for example to 0 or 3,

R₂ denotes a linear or branched C₁-C₁₄ alkyl or alkenyl radical, the hydrocarbon-based chain of which may be interrupted with a carbonyl unit and/or may be substituted with a phenyl group and/or a hydroxyl function.

According to one exemplary embodiment, the piperidine derivative according to the invention are chosen from: 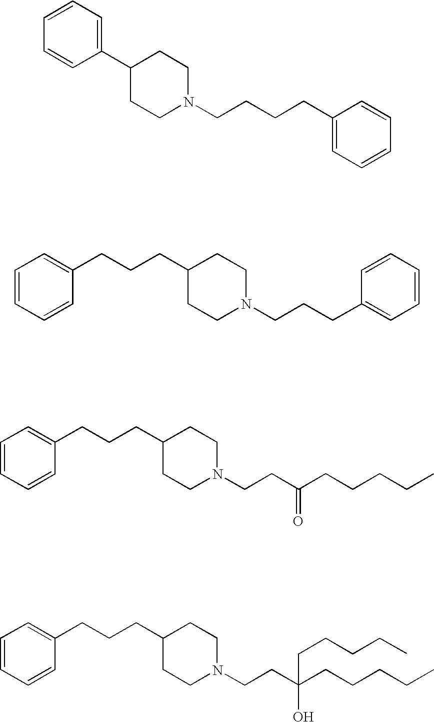
salts and isomers thereof, and mixtures thereof.

The compounds of formula (I) may for example be prepared according to the following reaction scheme: 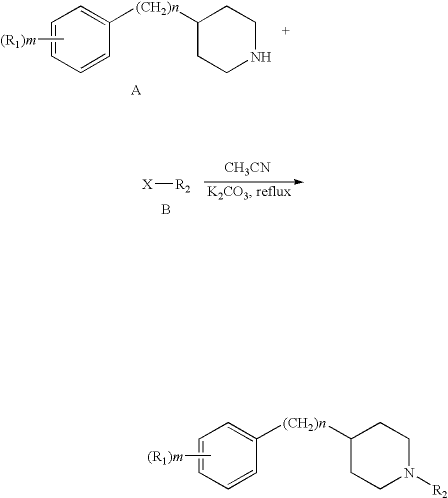
by reacting one equivalent of substituted piperidine A with one equivalent of B, in which X denotes a leaving group of halogen or sulfonate type, in the presence of K₂CO₃ in refluxing acetonitrile overnight. The product obtained may be worked up and purified on a column of silica.

The amount of piperidine derivative of general formula (I) or (II) that may be used according to the invention obviously depends on the desired effect and may thus vary within a wide range.

The effective amount corresponds to the amount required to obtain the desired result (i.e. in particular to increase the density of keratin fibers, to inhibit their loss and/or to promote their growth). A person skilled in the art is thus capable of evaluating this effective amount, which depends on the nature of the amine used, the person to whom it is applied and the time of this application.

To give an order of magnitude, these derivatives may be used in an amount representing from 0.001% to 10% of the total weight of the composition, for example in an amount representing from 0.01% to 5% of the total weight of the composition and for example in an amount representing from 0.1% to 2% of the total weight of the composition.

In the text hereinbelow, and unless otherwise mentioned, the amounts of the various ingredients in the composition are given as weight percentages relative to the total weight of the composition.

The compositions of the invention may be for cosmetic or pharmaceutical (for example dermopharmaceutical) use. The compositions of the invention may for example be for cosmetic use. The compositions must contain a non-toxic, physiologically acceptable medium that can be applied to the skin, including the scalp and the eyelids and to keratin fibers such as head hair and eyelashes.

The derivatives of formula (I), which may or may not be salified, may be used in a composition to be ingested, injected or applied to the skin or to keratin fibers (to any area of skin or fibers to be treated).

These compounds may also be administered orally in an amount of from 0.1 to 300 mg per day and for example from 5 to 10 mg/day.

According to one exemplary embodiment, a composition of the invention is a composition for cosmetic use and for example for topical application to the skin and keratin fibers, and for example to the scalp, the hair and the eyelashes;

This composition may be in any known presentation form that is suitable for the mode of use.

For topical application to the skin and keratin fibers, including the scalp, the composition may be in the form of an aqueous, alcoholic, aqueous-alcoholic or oily solution or suspension, an emulsion or dispersion of more or less fluid consistency and for example of liquid or semi-liquid consistency, obtained by dispersion of a fatty phase in an aqueous phase (O/W) or conversely (W/O), a solid (O/W) or (W/O) dispersion or emulsion, a more or less fluid or solid aqueous, aqueous-alcoholic or oily gel, a free or compacted powder to be used in unmodified form or to be incorporated into a physiologically acceptable medium, or alternatively microcapsules, microparticles or vesicular dispersions of ionic and/or nonionic type.

A composition in the form of a mousse or alternatively in the form of an aerosol or spray, then comprising a pressurized propellant, may also be envisaged.

The composition may thus be in the form of a lotion, a serum, a milk, an O/W or W/O cream, a gel, an ointment, a pomade, a powder, a balm, a patch, an impregnated pad, a soap, a bar or a mousse.

The composition for application to the scalp or the hair may be for example in the form of a haircare lotion, for example for daily or twice-weekly application, a shampoo or a hair conditioner, in particular for twice-weekly or weekly application, a liquid or solid scalp-cleansing soap for daily application, a hairstyle shaping product (lacquer, hair setting product or styling gel), a treatment mask, a foaming gel or cream for cleansing the hair. It may also be in the form of a hair dye or mascara to be applied with a brush or a comb.

Moreover, for application to the eyelashes or body hairs, the composition to which the invention applies may be in the form of a pigmented or unpigmented mascara, to be applied with a brush to the eyelashes or alternatively to beard or moustache hair.

For application to the nails, the compounds in accordance with the invention may be formulated in the form of a composition of varnish type.

For a composition for use by injection, the composition may be in the form of an aqueous lotion or an oily suspension, for example in the form of a serum. For oral use, the composition may be in the form of capsules, granules, drinkable syrups or tablets.

According to one exemplary embodiment, the composition according to the invention is in the form of a hair cream or hair lotion, a shampoo or hair conditioner, a hair mascara or an eyelash mascara.

The amounts of the various constituents of the physiological medium of the composition according to the invention are those generally used in the fields under consideration. In addition, these compositions may be prepared according to the usual methods.

When the composition is an emulsion, the proportion of the fatty phase may range from 2% to 80% by weight and for example from 5% to 50% by weight relative to the total weight of the composition. The aqueous phase is adjusted as a function of the content of fatty phase and of compound(s) (I) and also of that of the optional additional ingredients, to obtain 100% by weight. In practice, the aqueous phase represents from 5% to 99.9% by weight.

The fatty phase may contain fatty or oily compounds that are liquid at room temperature (25° C.) and atmospheric pressure (760 mm/Hg), which are generally known as oils. These oils may be mutually compatible or incompatible and may form a macroscopically homogeneous liquid fatty phase or a two-phase or three-phase system. In addition to these oils, the fatty phase may contain waxes, gums, lipophilic polymers or “pasty” or viscous products containing solid parts and liquid parts.

The aqueous phase contains water and optionally an ingredient that is miscible in all proportions with water, for instance C₁ to C₈ lower alcohols such as ethanol or isopropanol, polyols, for instance propylene glycol, glycerol or sorbitol, or alternatively acetone or ether.

The emulsifiers and co-emulsifiers used to obtain a composition in emulsion form are those generally used in cosmetics and pharmaceuticals. Their nature also depends on the sense of the emulsion. In practice, the emulsifier and, where appropriate, the co-emulsifier are present in the composition in a proportion ranging from 0.1% to 30% by weight, for example from 0.5 to 20% by weight and for example from 1% to 8% by weight. The emulsion may also contain lipid vesicles and for example liposomes.

When the composition is in the form of an oily solution or gel, the fatty phase may represent more than 90% of the total weight of the composition.

In one exemplary embodiment, for haircare use, the composition may be an aqueous, alcoholic or aqueous-alcoholic solution or suspension and for example a water/ethanol solution or suspension. The alcoholic fraction may represent from 5% to 99.9% and for example from 8% to 80%.

For use as a mascara, the composition may for example be a wax-in-water or wax-in-oil dispersion, a gelled oil or an aqueous gel, which may be pigmented or unpigmented.

The composition of the invention may also comprise other ingredients usually used in the fields under consideration, chosen from solvents, aqueous-phase or oily-phase thickeners or gelling agents, dyestuffs that are soluble in the medium of the composition, solid particles such as fillers or pigments, antioxidants, preserving agents, fragrances, electrolytes, neutralizers, UV blockers, for instance sunscreens, film-forming polymers, cosmetic and pharmaceutical active agents with a beneficial effect on the skin or keratin fibers, other than the compounds of formula (I) or (II) (such as vitamins) and mixtures thereof. These additives may be present in the composition in the amounts generally used in cosmetics and dermatology, and for example in a proportion of from 0.01% to 50% and for example from 0.1% to 20%, for example from 0.1% to 10%, relative to the total weight of the composition. Depending on their nature, these adjuvants may be introduced into the fatty phase, into the aqueous phase and/or into lipid vesicles and for example liposomes.

Needless to say, a person skilled in the art will take care to select the optional additional additives and/or the amount thereof such that the advantageous properties of the composition according to the invention, i.e. for example the increase in the density of keratin fibers, are not, or are not substantially, adversely affected by the envisaged addition.

As solvents that may be used in the invention, mention may be made of C₂ to C₈ lower alcohols, for instance ethanol, isopropanol, propylene glycol and certain light cosmetic oils, for instance C₆ to C₁₆ alkanes.

As oils that may be used in the invention, mention may be made of oils of mineral origin (liquid petroleum jelly or hydrogenated isoparaffin), oils of plant origin (liquid fraction of shea butter, sunflower oil, apricot oil, soybean oil, fatty alcohol or fatty acid), oils of animal origin (perhydrosqualene), synthetic oils (fatty acid esters, purcellin oil), silicone oils (linear or cyclic polydimethylsiloxanes, or phenyl trimethicones) and fluoro oils (perfluoropolyethers). Waxes that may be mentioned include silicone waxes, beeswax, candelilla wax, rice wax, carnauba wax, paraffin wax and polyethylene wax.

As emulsifiers that may be used in the invention, examples that may be mentioned include glyceryl stearate, glyceryl laurate, sorbitol stearate, sorbitol oleate, alkyl dimethicone copolyols (with alkyl≧8) and mixtures thereof for a W/O emulsion. Polyethylene glycol monostearate or monolaurate, polyoxyethylenated sorbitol stearate or oleate, and dimethicone copolyols, and mixtures thereof, may also be used for an O/W emulsion. The emulsifier and the co-emulsifier are present in the composition in a proportion ranging from 0.3% to 30% by weight and for example from 0.5% to 20% by weight relative to the total weight of the composition.

As hydrophilic gelling agents that may be used in the invention, mention may be made of carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents that may be used in the invention, mention may be made of modified clays, for instance Bentones, metal salts of fatty acids, for instance aluminium stearates, hydrophobic-treated silica and ethylcellulose, and mixtures thereof.

As cosmetic or pharmaceutical active agents other than the compounds of formula (I) or (II), the composition may contain an additional hydrophilic active agent chosen from proteins, protein hydrolysates, amino acids, polyols, urea, allantoin, sugars and sugar derivatives, water-soluble vitamins, plant extracts (those from Iridacea plants or from soybean) and hydroxy acids (fruit acid or salicylic acid); and/or an additional lipophilic active agent chosen from retinol (vitamin A) and its derivatives, for example an ester (retinyl palmitate), tocopherol (vitamin E) and its derivatives, for example an ester (tocopheryl acetate or palmitate), essential fatty acids, ceramides, essential oils, salicylic acid derivatives, for instance 5-n-octanoyl salicylic acid, hydroxy acid esters, and phospholipids, for instance lecithin, and mixtures thereof.

According to one exemplary embodiment of the invention, the compound of general formula (I) or a salt or isomer thereof may be combined with at least one additional compound that promotes the regrowth and/or limits the loss of keratin fibers (hair or eyelashes). These additional compounds are chosen for example from the lipoxygenase inhibitors as described in EP 0 648 488, the bradykinin inhibitors described for example in EP 0 845 700, prostaglandins and derivatives thereof, for example those described in WO 98/33497, WO 95/11003, JP 97-100 091 and JP 96-134 242, prostaglandin receptor agonists or antagonists, the non-prostanoic prostaglandin analogues as described in EP 1 175 891, EP 1 175 890, WO 01/74307, WO 01/74313, WO 01/74314, WO 01/74315 or WO 01/72268, and mixtures thereof.

As other additional compounds that promote the growth of hair, which may be present in the composition according to the invention, mention may be made of vasodilators, antiandrogens, cyclosporins and analogues thereof, antimicrobial and antifungal agents, anti-inflammatory agents, and retinoids, alone or as a mixture.

The vasodilators that may be used are for example potassium-channel agonists, including Minoxidil, and also the compounds described in patents U.S. Pat. Nos. 3,382,247, 5,756,092, 5,772,990, 5,760,043, 5,466,694, 5,438,058 and 4,973,474, cromakalim, nicorandil and diaxozide, alone or in combination.

The antiandrogens that may be used include for example steroidal and non-steroidal 5α-reductase inhibitors, for instance finasteride and the compounds described in U.S. Pat. No. 5,516,779, cyprosterone acetate, azelaic acid and the salts and derivatives thereof, and the compounds described in U.S. Pat. No. 5,480,913, flutamide, oxendolone, spironolactone, diethylstilbestrol and the compounds described in patents U.S. Pat. Nos. 5,411,981, 5,565,467 and 4,910,226.

The antimicrobial or antifungal compounds may be chosen from selenium derivatives, octopirox, triclocarban, triclosan, zinc pyrithione, itraconazole, asiatic acid, hinokitiol, mipirocine, tetracyclines, for example erythromycin and the compounds described in EP 0 680 745, clinycin hydrochloride, benzoyl peroxide or benzyl peroxide, minocycline and compounds belonging to the imidazole class, such as econazole, ketoconazole or miconazole or salts thereof, nicotinic acid esters, including for example tocopheryl nicotinate, benzyl nicotinate and C₁-C₆ alkyl nicotinates, for instance methyl or hexyl nicotinate.

The anti-inflammatory agents may be chosen from steroidal anti-inflammatory agents, for instance glucocorticoids, corticosteroids (for example: hydrocortisone) and non-steroidal anti-inflammatory agents, for instance glycyrrhetinic acid and α-bisabolol, benzydamine, salicylic acid and the compounds described in EP 0 770 399, WO 94/06434 and FR 2 268 523.

The retinoids may be chosen from isotretinoin, acitretin and tazarotene.

As other additional active compounds for promoting the growth and/or limiting the loss of hair that may be used in combination with the compound of formula (I), which may or may not be salified, mention may be made of aminexil, 6-O-[(9Z,12Z)octadeca-9,12-dienoyl]hexapyranose, benzalkonium chloride, benzethonium chloride, phenol, oestradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharide or acylhexosaccharide acids, substituted aryl ethylenes, N-acylamino acids, flavonoids, ascomycin derivatives and analogues, histamine antagonists, saponins, proteoglycanase inhibitors, oestrogen agonists and antagonists, pseudoterines, cytokines and growth factor promoters, IL-1 or IL-6 inhibitors, IL-10 promoters, TNF inhibitors, benzophenones and hydantoin, retinoic acid; vitamins, for instance vitamin D, vitamin B12 analogues and pantothenol; triterpenes, for instance ursolic acid and the compounds described in U.S. Pat. No. 5,529,769, U.S. Pat. No. 5,468,888 and U.S. Pat. No. 5,631,282; antipruriginous agents, for instance thenaldine, trimeprazine or cyproheptadine; antiparasitic agents, for example metronidazole, crotamiton or pyrethroids; calcium antagonists, for instance cinnarizine, diltiazem, nimodipine, verapamil, alverine and nifedipine; hormones such as oestriol or its analogues, thyroxine and its salts, or progesterone; PF receptor (type-F prostaglandin receptor) antagonists such as latanoprost, bimatoprost, travoprost or unoprostone; 15-hydroxyprostaglandine dehydrogenase inhibitors; mixtures thereof.

It may also be envisaged for the composition comprising at least one compound of general formula (I), which may or may not be salified, to be in liposomal form, as described for example in document WO 94/22468. Thus, the compound encapsulated in the liposomes may be delivered selectively to the hair follicle.

The composition to which the invention applies may be applied to the alopecic areas of the scalp and the hair of an individual, and optionally left in contact for several hours and optionally rinsed out.

The composition containing an effective amount of a compound of general formula (I), which may or may not be salified, may, for example, be applied in the evening, kept in contact throughout the night and optionally shampooed out in the morning. These applications may be repeated daily for one or more months according to the individual.

The treatment process according to the invention may have the characteristics of a cosmetic process since it makes it possible to enhance the appearance of keratin fibers and in particular the hair and the eyelashes, by giving them greater vigour and an improved look. In addition, it may be used daily for several months, without medical prescription.

In the process according to the invention, between 5 and 500 μl of a solution or composition as defined above, comprising from 0.001% to 5% by weight of compound of general formula (I), may be for example applied to the areas of the scalp to be treated.

Other characteristics and advantages of the invention will emerge more clearly from the examples that follow, which are given as non-limiting illustrations. In the text hereinbelow, the proportions are given as mass percentages, unless otherwise indicated.

### EXAMPLE 1

The compounds presented in Table I were prepared according to the protocols discussed below.

| | |
|---|---|
| 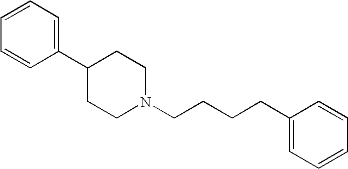 | Compound 1 |
| 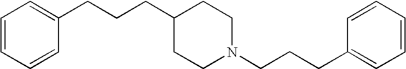 | Compound 2 |
| 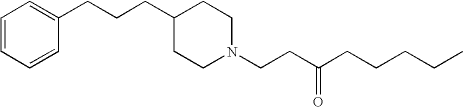 | Compound 3 |
| 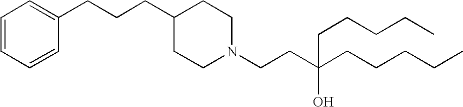 | Compound 4 |

1) Synthesis of Compounds 1 and 2 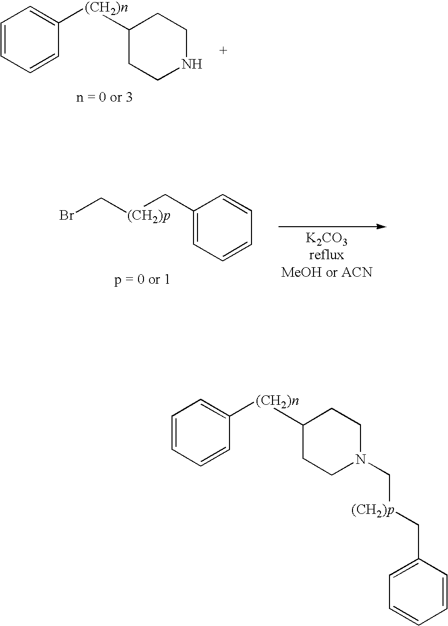
The corresponding piperazine is placed in contact with the corresponding alkyl bromide (1 eq.) in the presence of K₂CO₃ in refluxing methanol or acetonitrile overnight. After the reaction, the expected product is recovered and purified on a column of silica. The 500 MHz ¹H NMR spectrum confirms its chemical structure.

2) Synthesis of compound 3 and compound 4 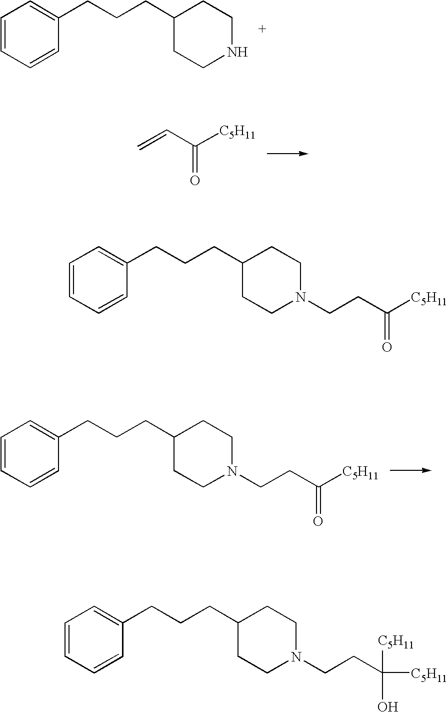
1-(3-Phenylpropyl)piperidine (1 eq.) and 1-octen-3-one (1 eq.) are placed in contact in ethanol at room temperature. The expected product (compound 3) is recovered and purified on a column of silica. NMR and MS analyses confirm its chemical structure.

The intermediate ketone (1 eq.) corresponding to compound 3 is reacted with bromopentylmagnesium (1 eq.) in anhydrous THF under nitrogen, at room temperature. Compound 4 thus obtained is recovered and purified on a column of silica. NMR and MS analyses confirm its chemical structure.

### EXAMPLE 2

Compounds 1 and 2 were tested at a given concentration, on a model of nerve/muscle coculture, which makes it possible to recreate a motor arc by innervating human striated muscle cells with explants of rat embryonic spinal ganglia and spinal cord.

This test is predictive of an inhibitory effect on muscle fiber contractions.

The protocol adopted is as follows:

Human myoblasts (primary culture of human muscle cells, M1b) were inoculated in 24-well plates. The culture medium is a mixture of ⅔ MEM (Invitrogen 21090-022), ⅓ M199 (Invitrogen 31153-026) and 5% FCS. At confluence, spinal cord explants from 13-day-old rat embryos are placed on the primary culture. The first contractions of the muscle fibers are observed after one week of coculture. After three weeks, these muscle fibers are striated and have mature differentiated neuromuscular junctions.

The products are tested in triplicate (3 fibers analysed) at two concentrations. For each culture well, a muscle fiber having regular contractions (>60 contractions per minute) is selected and the number of contractions is counted over 30 seconds. The test product is then added to the culture medium under a microscope without moving the microscope stage. After incubation for 60 seconds, the number of contractions is again counted over 30 seconds. The whole experiment is recorded using a digital camera. The videos are saved on an electronic medium.

Compound 1 induces a dose-response effect with inhibition of 50% of the frequency of contraction of the muscle fibers at a concentration of 10⁻⁵ M. Compound 2 shows inhibition of about 55% at a concentration of 10⁻⁵ M.

### EXAMPLE 3

| Hair lotion: | Weight % |
|---|---|
| Compound 1 | 1 |
| Propylene glycol | 30 |
| Ethyl alcohol | 40 |
| Water qs | 100 |

### EXAMPLE 4

| Wax/water mascara: | Weight % |
|---|---|
| Beeswax | 6 |
| Paraffin wax | 13 |
| Hydrogenated jojoba oil | 2 |
| Water-soluble film-forming polymer | 3 |
| Triethanolamine stearate | 8 |
| Compound 2 | 1 |
| Black pigment | 5 |
| Preserving agent qs | |
| Water qs | 100 |

This mascara is applied to the eyelashes like a standard mascara with a mascara brush.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A process for treating human keratin fibers and/or skin from which the fibers emerge to induce growth, stimulate growth, reduce loss and/or increase density of the fibers, comprising:
applying a cosmetic composition to the fibers and/or skin;
leaving the cosmetic composition in contact with the fibers and/or skin; and
optionally rinsing the fibers and/or skin;
wherein:
the cosmetic composition comprises an effective amount of at least one of a piperidine derivative, a salt of the piperidine derivative, and an isomer of the piperidine derivative;
the piperidine derivative is given by the general formula (I) 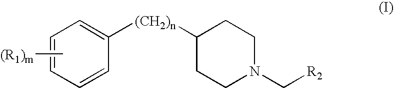
where:
R₁ is a halogen or a radical selected from the group consisting of a linear or branched C₁-C₆ alkyl radical, an OR group, an NRR′group, a CF₃ group, an NHCOR group or a CONRR′group;
R₂ is a linear or branched C₁-C₂₀ alkyl or alkenyl radical;
m is an integer from 0 to 5; and
n is an integer from 0 to 5.

2. The process according to claim 1, wherein R₂ is a linear or branched C₁-C₂₀ alkyl or alkenyl radical, the hydrocarbon-based chain of which is interrupted with a —CO-function.

3. The process according to claim 1, wherein:
R₂ is a linear or branched C₁-C₂₀ alkyl or alkenyl radical, the hydrocarbon-based chain of which is substituted with at least one of an OR group, a COOR group, a NRR′ group, a NHCOR group, a CONRR′ group and a phenyl group; and
R and R′ are each independently a hydrogen atom or a linear or branched C₁-C₆ alkyl radical.

4. The process according to claim 3, wherein the phenyl group is substituted with one or more radicals R₁.

5. The process according to claim 1 wherein:
m is an integer from 0 to 3;
n is an integer from 0 to 3;
R₂ is a linear or branched C₁-C₁₄ alkyl or alkenyl radical.

6. The process according to claim 5, wherein m is equal to 0.

7. The process according to claim 5, wherein n is equal to 0 or 3.

8. The process according to claim 5, wherein R₂ is a linear or branched C₁-C₄ alkyl or alkenyl radical, the hydrocarbon-based chain of which is at least one of: interrupted with a carbonyl unit, substituted with a phenyl group, and substituted with a hydroxyl function.

9. The process according to claim 1, wherein the piperidine derivative is selected from the group consisting of:

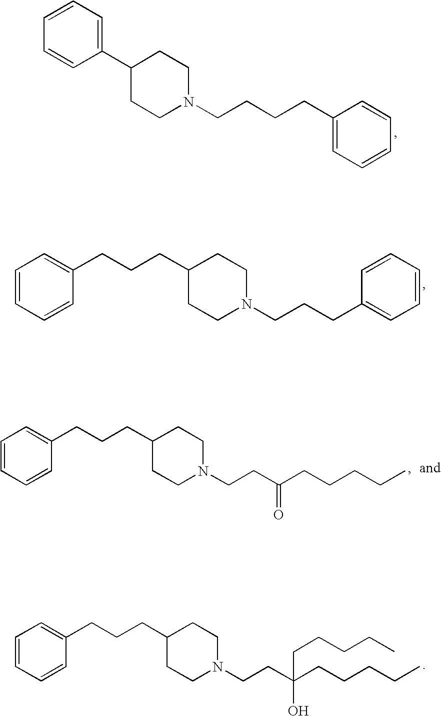

10. The process according to claim 1, wherein the at least one of the piperidine derivative, the salt of the piperidine derivative, and the isomer of the piperidine derivative is present in the cosmetic composition in an amount of from 10⁻³ to 10% by weight relative to a total weight of the cosmetic composition.

11. The process according to claim 1, wherein the cosmetic composition is in the form of a hair cream, a hair lotion, a shampoo, a hair conditioner, a hair mascara or an eyelash mascara.

12. The process according to claim 11, wherein the cosmetic composition is in the form of an aqueous, alcoholic or aqueous-alcoholic solution or suspension.

13. The process according to claim 1, wherein the composition further comprises at least one ingredient selected from the group consisting of solvents, aqueous-phase or oily-phase thickeners or gelling agents, dyestuffs that are soluble in the composition, fillers, pigments, antioxidants, preserving agents, fragrances, electrolytes, neutralizers, film-forming polymers, UV blockers, cosmetic agents, and pharmaceutically active agents.

14. The process according to claim 1, wherein the cosmetic composition further comprises at least one additional active compound that promotes regrowth and/or limits loss of the fibers.

15. The process according to claim 14, wherein the additional active compound is selected from the group consisting of aminexil, 6-O-[(9Z,12Z)octadeca-9,12-dienoyl]hexapyranose, lipoxygenase inhibitors, bradykinin inhibitors, prostaglandins, prostaglandin derivatives, prostaglandin receptor agonists or antagonists, non-prostanoic prostaglandin analogues, vasodilators, antiandrogens, cyclosporins, cyclosporin analogues, antimicrobial agents, anti-inflammatory agents, retinoids, benzalkonium chloride, benzethonium chloride, phenol, oestradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharide or acylhexosaccharide acids, substituted aryl ethylenes, N-acylamino acids, flavonoids, ascomycin derivatives and analogues, histamine antagonists, saponins, proteoglycanase inhibitors, oestrogen agonists and antagonists, pseudoterines, cytokines and growth factor promoters, L-1 or IL-6 inhibitors, IL-10 promoters, TNF inhibitors, vitamins, benzophenones, hydantoin, retinoic acid, antipruriginous agents, antiparasitic agents, antifungal agents, calcium antagonists, hormones, triterpenes, antiandrogens, steroidal or non-steroidal 5α-reductase inhibitors, potassium-channel agonists, and PF receptor antagonists.

16. The process according to claim 1, wherein the cosmetic composition further comprises at least one active agent selected from the group consisting of proteins, protein hydrolysates, amino acids, polyols, urea, allantoin, sugars and sugar derivatives, water-soluble vitamins, plant extracts, hydroxy acids, retinol, tocopherol, retinol or tocopherol derivatives, essential fatty acids, ceramides, essential oils, salicylic acid derivatives, for instance 5-n-octanoylsalicylic acid, hydroxy acid esters, and phospholipids.

17. A process for treating disorders associated with a reduction in cutaneous capillary circulation or vascularization, comprising:
applying a cosmetic composition to human keratin fibers and/or skin;
leaving the cosmetic composition in contact with the fibers and/or skin; and
optionally rinsing the fibers and/or skin;
wherein:
the cosmetic composition comprises an effective amount of at least one of a piperidine derivative, a salt of the piperidine derivative, and an isomer of the piperidine derivative;
the piperidine derivative is given by the general formula (I) 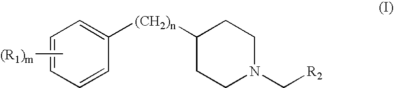
where:
R₁ is a halogen or a radical selected from the group consisting of a linear or branched C₁-C₆ alkyl radical, an OR group, an NRR′group, a CF₃ group, an NHCOR group or a CONRR′group;
R₂ is a linear or branched C₁-C₂₀ alkyl or alkenyl radical;
m is an integer from 0 to 5; and
n is an integer from 0 to 5.

18. A process for treating andro-chrono-genetic alopecia, comprising
applying a cosmetic composition to human hair and/or scalp;
leaving the cosmetic composition in contact with the hair and/or scalp; and
optionally rinsing the hair and/or scalp;
wherein:
the cosmetic composition comprises an effective amount of at least one of a piperidine derivative, a salt of the piperidine derivative, and an isomer of the piperidine derivative;
the piperidine derivative is given by the general formula (I) 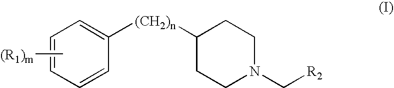
where:
R₁ is a halogen or a radical selected from the group consisting of a linear or branched C₁-C₆ alkyl radical, an OR group, an NRR′group, a CF₃ group, an NHCOR group or a CONRR′group;
R₂ is a linear or branched C₁-C₂₀ alkyl or alkenyl radical;
m is an integer from 0 to 5; and
n is an integer from 0 to 5.

19. A process for caring for and/or making human eyelashes, comprising applying a cosmetic composition to the eyelashes and/or upper eyelids, wherein:
the cosmetic composition comprises an effective amount of at least one of a piperidine derivative, a salt of the piperidine derivative, and an isomer of the piperidine derivative;
the piperidine derivative is given by the general formula (I) 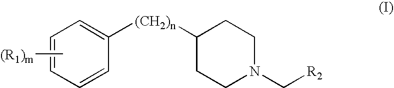
where:
R₁ is a halogen or a radical selected from the group consisting of a linear or branched C₁-C₆ alkyl radical, an OR group, an NRR′group, a CF₃ group, an NHCOR group or a CONRR′group;
R₂ is a linear or branched C₁-C₂₀ alkyl or alkenyl radical;
m is an integer from 0 to 5; and
n is an integer from 0 to 5.
